(19) United States
(12) Patent Application Publication
(10) Pub. No.: US 2009/0298070 A1
(43) Pub. Date: Dec. 3, 2009

(54) METHOD FOR ANALYZING METABOLITES FLUX USING CONVERGING RATIO DETERMINANT AND SPLIT RATIO DETERMINANT

(75) Inventors: Sang-Yup Lee, Daejeon (KR); Hyung Seok Choi, Daejeon (KR); Tae Yong Kim, Gyeonggi-do (KR)

Correspondence Address: MOORE & VAN ALLEN PLLC, P.O. BOX 13706, Research Triangle Park, NC, 27709, US

(73) Assignee: Korea Advanced Institute of Science and Technology, Daejeon (KR)

(21) Appl. No.: 12/297,343

(22) PCT Filed: Sep. 8, 2006
(86) PCT No.: PCT/KR2006/003578

§ 371 Date: Mar. 13, 2009

(30) Foreign Application Priority Data
Apr. 21, 2006 (KR) 1020060036128

(51) Int. Cl. C12Q 1/68; C12Q 1/02; C12P 19/34

(52) U.S. Cl. 435/6; 435/29; 435/91.1

(57) ABSTRACT

The present invention relates to a method for analyzing metabolic flux using CRD and SRD. Specifically, the method comprising: selecting a specific target organism, constructing the metabolic network model of the selected organism, identifying the correlations between specific metabolic fluxes in the metabolic network model, defining the correlation ratios as CRD and SRD, determining the correlation ratios of the metabolic fluxes through the experiment for measuring metabolic flux ratios, modifying a stoichiometric matrix with the determined CRD, SRD and correlation ratios, and applying the modified stoichiometric matrix of the metabolic network model for linear programming. According to the inventice method, the correlation between influent/effluent metabolic fluxes with respect to specific metabolites in target organisms (including E. coli), the genome-scale metabolic network model of which was constructed, can be determined as relative ratio using useful information obtained from various experiments, including a growth experiment using a radioactive isotope-labeled carbon source and an assay for measuring enzymatic reaction. Thus, limit values from various experiments can be effectively applied, so that internal metabolic flux can be quantified and analyzed in a more accurate and rapid manner.

### TECHNICAL FIELD

The present invention relates to a method for 54 analyzing intracellular metabolic flux using CRD and SRD, and more particularly to a metabolic flux analysis method comprising: selecting a specific target organism, constructing the metabolic network model of the selected organism based on the biochemical reactions, identifying the correlations between specific metabolic fluxes in the metabolic network model, defining the correlations as CRD and SRD, determining the flux ratios of the specific biochemical reactions in the metabolic network model through a ¹³C labeling experiments, modifying a stoichiometric matrix with the determined CRD, SRD and artificial metabolites, and applying the modified stoichiometric matrix to a metabolic network model for linear programming.

### BACKGROUND ART

Metabolic engineering provide information required to alter the metabolic characteristics of cells or strains in the direction we desire, by introducing new biochemical reactions or removing, amplifying or modifying the existing metabolic pathways using molecular biological technology related to the genetic recombinant technology. Such metabolic engineering include the overall contents of bioengineering, such as the overproduction of existing metabolites, the production of new metabolites, the suppression of production of undesired metabolites, and the utilization of inexpensive substrates. With the aid of increasing bioinformatics newly developed therewith, it became possible to construct each metabolic network model from the genomic information of various species. By the combination of the metabolic network information and the metabolic flux analysis technology, industrial application possibilities of the production of various primary metabolites and useful proteins are now shown (Hong et al., Biotech. Bioeng, 83:854, 2003; US 2002/0168654; Price et al., Nat. Rev. Microbiol., 2:886, 2004).

Generally, among the techniques of metabolic flux analysis for analyzing cell metabolism, methods based on a static model that considers only the coefficient of a simple biochemical scheme can be broadly divided into two methods: constraints-based flux analysis based on linear programming; and a metabolic flux analysis method based on non-linear programming. The constraints-based flux analysis based on linear programming has easy accessibility and simple calculation procedures, but has a problem in that the number of usable constraints is small, making it impossible to obtain real values. Also, the method based on non-linear programming can use a large number of constraints, and thus can provide more exact flux values, but has problems in that it restricts the scale of metabolic network model and requires complex calculation procedures, difficult experimental procedures, and also requires much time for calculation (Varma et al., Bio-Technol., 12:994, 1994; Nielsen et al., Bioreaction Engineering Principles, Plenum Press, 1994; Lee et al., Metabolic Engineering, Marcel Dekker, 1999; K. Shimizu, Biotechnol. Bioprocess Eng., 7:237, 2002).

The metabolic flux analysis is a technology to quantify metabolic fluxes in an organism. The metabolic flux analysis is based on the assumption of a pseudo-steady state. Namely, since a change in the concentration of internal metabolites caused by a change in external environment is very immediate, this change is generally neglected and it is assumed that the concentration of internal metabolites is not changed.

If all metabolites, biochemical pathways and the stoichiometric matrix in the pathways (Sᵢⱼ^{T}, metabolite i in the j reaction) are known, the metabolic flux vector (vⱼ, flux of j pathway) can be calculated, in which a change in the metabolite X with time can be expressed as the sum of all metabolic fluxes. Assuming that a change in X with time is constant i.e., under the assumption of the quasi-steady state, the following equation is defined:

S^{T}v=dX/dt=0

However, there are many cases where only pathways are known and stoichiometric value for each metabolite and pathway and the metabolic flux vector (vⱼ) are partially known, and thus, the above equation is expanded to the following equation:

S^{T}v=Sₘvₘ+Sᵤvᵤ0=

The above equation is divided into two matrices; a defined matrix of experimentally known stoichiometric value (Sₘ(I×M), I=total metabolite number, M=total stoichiometrically-known reaction number) times flux (vₘ(M×I)) and a matrix of unknown stoichiometric value (Sᵤ(I×M)) times flux (vᵤ(M×I)). In this regard, m is a subscript for measurement value, and u is a subscript for unmeasurable value.

If the rank (Sᵤ) of the unknown flux vector is equal to u (i.e., if the number of variables is equal to an equation), flux is then determined from a simple matrix calculation. However, if the rank (Sᵤ) of the unknown flux vector is greater than u (i.e., if a superposed equation exists), operations for verifying the consistency of total equations, accuracy for the measurement values of metabolic flux, and the validity of a quasi-steady state, will be performed for the calculation of more accurate values, followed by determining metabolic flux using non-linear programming, such as parameter assumption approximation methods, etc.

If the number of variables is greater than an equation, the optimal metabolic flux distribution is then calculated by linear programming using specific objective functions and various physicochemical equations where the flux value of a specific metabolic reaction can be limited to a specific range. This can be calculated as follows:

minimize/maximize:Z=Σcᵢvᵢ

s.t. S^{T}v=0 and α_{min,i}≦vᵢ≦α_{max,I}

wherein cᵢ is weighted value, and vᵢ is metabolic flow.

Generally, the maximization of biomass formation rate (i.e., specific growth rate), the maximization of typical metabolite production and the minimization of byproduct production, and the like, are used as the objective functions. α_{max,i} and α_{min,i} are limit values which each metabolic flux can have, and they can assign the maximum and minimum values permissible in each metabolic flux.

Metabolic flux analysis techniques based on this linear programming method provide a variety of information, and thus are very useful, but in most cases, actual metabolic flux network models have the number of parameters significantly larger than the number of equations, and thus cannot provide real values for internal metabolic flux values.

For this reason, a technique for analyzing intracellular metabolic flux has recently been developed, in which the isotope contents of major metabolites from cell growth experiments using a radioactive isotope-labeled carbon source are measured by GCMS (gas chromatography-mass spectrometry) and used as additional constraints. This technique, also called isotopomer analysis, has an advantage in that it performs calculations on the basis of the non-linear programming method and provides actual metabolic flux distributions for central metabolic networks. However, this technique has disadvantages in that, because it is based on the non-linear programming method, it uses a very complex calculation procedure that gives a user inconvenience and long calculation time, and also it can perform calculation only in small-scale models, including glycolysis, pentosphosphate pathway, TCA, anaplerotic pathway and several amino acid synthetic pathways, due to the complexity thereof and a limitation in the range and number of constraints (Biotechnol. Bioeng. 66:86, 1999; K. Shimizu, Biotechnol. Bioprocess Eng. 7:237, 2002).

Accordingly, the present inventors have made many efforts to analyze metabolic flux in a more accurate and easy manner and, as a result, found that metabolic flux analysis for analyzing the metabolic characteristics of a target organism can be obtained in an accurate and rapid manner by determining the flux ratios of specific metabolic reactions through data of various experiments, including a growth experiment using a radioactive isotope-labeled carbon source and an experiment for the measurement of enzymatic reaction rate, applying the flux ratios to a stoichiometric matrix using the concepts of CRD, SRD, and artificial metabolites, and performing metabolic flux analysis using the stoichiometric matrix based on Linear programming, thereby completing the present invention.

### SUMMARY OF THE INVENTION

It is an object of the present invention to provide a method for metabolic flux analysis comprising the steps of: selecting a specific target organism, constructing the metabolic network model of the selected organism, defining the correlation of specific metabolic fluxes as CRD and SRD, determining the flux ratios of the metabolic fluxes through experiments such as 13C labeling experiment or enzyme activity assay, and plotting the profile of the overall metabolic flux of the metabolic network based on linear programming with concepts of CRD, SRD, and artificial metabolites.

Another object of the present invention is to provide a method for analyzing the metabolic flux of E. coli, comprising said steps.

To achieve the above objects, the present invention provides a method for analyzing the flux distributions of a target organism, the method comprising the steps of: (a) selecting a target organism (except for human beings), constructing the metabolic network model of the selected organism, and the overall metabolic flux of which is to be measured; (b) identifying the correlations between specific metabolic fluxes in the metabolic network model of the step (a), and defining the correlation of the specific metabolic fluxes as CRD (converging ratio determinant) and SRD (split ratio determinant); (c) performing a various experiment on the selected organism such as 13C labeling experiment or enzyme activity assay, and determining and correcting the flux ratios, CRD and SRD of the step (b) on the basis of the experimental results; (d) defining artificial metabolites between the specific metabolic fluxes; (e) determining additional equations by applying the corrected flux ratios, CRD and SRD of the step (c) to a stoichiometric matrix for solving linear equations using the artificial metabolites of the step (d), and applying the determined additional equations to the stoichiometric matrix to modify the stoichiometric matrix; and (f) plotting the optimal value and profile of the overall metabolic flux of the metabolic network according to linear programming using the stoichiometric matrix modified in the step (e).

In the present invention, the target organism is preferably a microorganism. Also, the correlation ratios of the specific metabolic fluxes in the step (b) are preferably defined as CRD and SRD using the following equations 1 and 2:

$$\begin{matrix}{C}_{q} = \frac{{\sum }_{p ∈ P} {f}_{p} - {f}_{q}}{{f}_{q}} , ∀ q ∈ P & [ Equation 1 ] \\ {D}_{q} = \frac{{\sum }_{p ∈ P} {f}_{p} - {f}_{q}}{{f}_{q}} , ∀ q ∈ P & [ Equation 2 ]\end{matrix}$$

wherein fₚ is the correlation ratio of specific metabolic flux p, f_{q} is the correlation ratio of specific metabolic flux q, C_{q} is CRD for specific metabolic flux v_{q} in converging pathways, D_{q} is SRD for specific metabolic flux v_{q} in split pathways (provided that

$${\sum }_{p ∈ P} {f}_{p} = 1 , ∀ q ∈ P$$

wherein P represents all the converging pathways or split pathways).

Also, the experiment for metabolic flux ratio in the step (c) is preferably a growth experiment using a radioactive isotope-labeled carbon source and/or an assay of enzyme reactions.

Moreover, the artificial metabolites in the step (d) are preferably defined using the following equation 3:

$$\frac{d {M}_{arf}^{CRD}}{d t} = {input}_{{M}_{arf}} - {output}_{{M}_{arf}} = {v}_{q} - {C}_{q} ( {\sum }_{p ∈ P} {v}_{p} - {v}_{q} ) = 0$$

$$\frac{d {M}_{arf}^{SRD}}{d t} = {input}_{{M}_{arf}} - {output}_{{M}_{arf}} = {v}_{q} - {D}_{q} ( {\sum }_{p ∈ P} {v}_{p} - {v}_{q} ) = 0$$

wherein M_{arf}^{CRD} is an artificial metabolite for CRD in converging pathways, M_{arf}^{SRD} is an artificial metabolite for SRD in split pathways, input_{Marf} is the sum of metabolic fluxes flowing into artificial metabolite M, output_{Marf} is the sum of metabolic fluxes flowing from artificial metabolite M, vₚ and v_{q} are the metabolic flux rates of metabolic fluxes p and q, respectively, C_{q} is CRD for specific metabolic flux q, and D_{q} is SRD for specific metabolic flux v_{q}.

Furthermore, the stoichiometric matrix before modification in the step (e) is preferably represented as follows:

$$S \cdot v = [ \begin{matrix}1 & - 1 & 0 & - 1 & 0 & 0 \\ 0 & 1 & - 1 & 0 & - 1 & 0 \\ 0 & 0 & 0 & 1 & 1 & - 1\end{matrix} ] \cdot [ \begin{matrix}v 1 \\ v 2 \\ v 3 \\ v 4 \\ v 5 \\ v 6\end{matrix} ] = 0$$

Also, the stoichiometric matrix after modification in the step (e) is preferably represented as follows:

$$S \cdot v = [ \begin{matrix}1 & - 1 & 0 & - 1 & 0 & 0 \\ 0 & 1 & - 1 & 0 & - 1 & 0 \\ 0 & 0 & 0 & 1 & 1 & - 1 \\ 0 & 0 & 0 & 1 & - {C}_{q} & 0\end{matrix} ] \cdot [ \begin{matrix}v 1 \\ v 2 \\ v 3 \\ v 4 \\ v 5 \\ v 6\end{matrix} ] = 0$$

In addition, the optimal value and profile of the overall metabolic flux in the step (f) are preferably plotted using the following equation 4:

$$Maximize / minimize \text{:} Z = {\sum }_{j ∈ J} {c}_{j} {v}_{j} \\ Subject to \begin{matrix}{\sum }_{j ∈ J} {S}_{ij} {v}_{j} = {b}_{i} & ∀ i ∈ I \\ {l}_{i} \leq {\sum }_{j ∈ J} {S}_{ij} {v}_{j} = {u}_{i} & ∀ i ∈ E \\ {a}_{j} \leq {v}_{j} \leq {β}_{j} & ∀ j ∈ J\end{matrix} \qquad [ Equation 4 ]$$

wherein Sᵢⱼ is the stoichiometric coefficient of the i^{th} metabolite in the j^{th} reaction, vⱼ is a metabolic flux vector in the j^{th} pathway, I is a set of all metabolites, J is a set of metabolic fluxes in all pathways, E is a set of all influent/effluent metabolic fluxes, cⱼ is a weighted value for a metabolic flux for the j^{th} pathway, bᵢ is the net transport flux of the i^{th} metabolite, lᵢ and uᵢ denote the net transport flux of the i^{th} metabolite, and αⱼ and βⱼ are limit values, which can be processed by metabolic flux in the j^{th} pathway.

In another aspect, the present invention provides a method for screening a gene to be amplified or deleted for increasing the production of a useful substance, the method comprising using the optimal value and profile of the overall metabolic flux of a metabolic network, plotted according to the above-described analysis method.

In still another aspect, the present invention provides a method for improving an organism producing a useful substance, the method comprising amplifying or deleting said screened gene in a target organism.

In yet another aspect, the present invention provides a method for analyzing the metabolic flux of E. coli, the method comprising the steps of: (a) constructing a metabolic network model of E. coli; (b) identifying the correlations between specific metabolic fluxes in the metabolic network model constructed in the step (a), and defining the correlations between the specific metabolic fluxes as CRD (converging ratio determinant) and SRD (split ratio determinant); (c) performing a experiment for the measurement of metabolic flux ratio on the E. coli, and determining and correcting the correlation ratios, CRD and SRD of the step (b) on the basis of the experimental results; (d) defining artificial metabolites between the specific metabolic fluxes; (e) determining additional equations by applying the corrected correlation ratios, CRD and SRD of the step (c) to a stoichiometric matrix for solving linear equations using the artificial metabolites of the step (d), and applying the determined additional equations to the stoichiometric matrix to modify the stoichiometric matrix; and (f) plotting the optimal value and profile of the overall metabolic flux of the metabolic network according to linear programming using the stoichiometric matrix modified in the step (e).

In the present invention, the correlation ratios of the specific metabolic fluxes in the step (b) are preferably defined as CRD and SRD using the following equations 1 and 2:

$$\begin{matrix}{C}_{q} = \frac{{\sum }_{p ∈ P} {f}_{p} - {f}_{q}}{{f}_{q}} , ∀ q ∈ P & [ Equation 1 ] \\ {D}_{q} = \frac{{\sum }_{p ∈ P} {f}_{p} - {f}_{q}}{{f}_{q}} , ∀ q ∈ P & [ Equation 2 ]\end{matrix}$$

wherein fₚ is the correlation ratio of specific metabolic flux p, f_{q} is the correlation ratio of specific metabolic flux q, C_{q} is CRD for specific metabolic flux v_{q} in converging pathways, D_{q} is SRD for specific metabolic flux v_{q} in split pathways (provided that

$${\sum }_{p ∈ P} {f}_{p} = 1 , ∀ p ∈ P$$

wherein P represents all the converging pathways or split pathways).

Also, the experiment for metabolic flux ratio in the step (c) is preferably a growth experiment using a radioactive isotope-labeled carbon source and/or an assay of enzyme reactions.

Moreover, the artificial metabolites in the step (d) are preferably defined using the following equation 3:

$$\begin{matrix}\frac{d {M}_{artf}^{C R D}}{d t} = {input}_{{M}_{artf}} - {output}_{{M}_{artf}} \\ = {v}_{q} - {C}_{q} ( {\sum }_{p ∈ P} {v}_{p} - {v}_{q} ) \\ = 0\end{matrix} \\ \begin{matrix}\frac{d {M}_{artf}^{S R D}}{d t} = {input}_{{M}_{artf}} - {output}_{{M}_{artf}} \\ = {v}_{q} - {D}_{q} ( {\sum }_{p ∈ P} {v}_{p} - {v}_{q} ) \\ = 0\end{matrix} \qquad [ Equation 3 ]$$

wherein M_{arf}^{CRD} is an artificial metabolite for CRD in converging pathways, M_{arf}^{SRD} is an artificial metabolite for SRD in split pathways, input_{Marf} is the sum of metabolic fluxes flowing into artificial metabolite M, output_{Marf} is the sum of metabolic fluxes flowing from artificial metabolite M, vₚ and v_{q} are the metabolic flux rates of metabolic fluxes p and q, respectively, C_{q} is CRD for specific metabolic flux q, and D_{q} is SRD for specific metabolic flux v_{q}.

Furthermore, the stoichiometric matrix before modification in the step (e) is preferably represented as follows:

$$S \cdot v = [ \begin{matrix}1 & - 1 & 0 & - 1 & 0 & 0 \\ 0 & 1 & - 1 & 0 & - 1 & 0 \\ 0 & 0 & 0 & 1 & 1 & - 1\end{matrix} ] \cdot [ \begin{matrix}v 1 \\ v 2 \\ v 3 \\ v 4 \\ v 5 \\ v 6\end{matrix} ] = 0$$

Also, the stoichiometric matrix after modification in the step (e) is preferably represented as follows:

$$S \cdot v = [ \begin{matrix}1 & - 1 & 0 & - 1 & 0 & 0 \\ 0 & 1 & - 1 & 0 & - 1 & 0 \\ 0 & 0 & 0 & 1 & 1 & - 1 \\ 0 & 0 & 0 & 1 & - {C}_{q} & 0\end{matrix} ] \cdot [ \begin{matrix}v 1 \\ v 2 \\ v 3 \\ v 4 \\ v 5 \\ v 6\end{matrix} ] = 0$$

In addition, the optimal value and profile of the overall metabolic flux in the step (f) are plotted using the following equation 4:

$$Maximize \text{/} minimize \text{:} Z = {\sum }_{j ∈ J} {c}_{j} {v}_{j} \\ Subject to \\ \begin{matrix}{\sum }_{j ∈ J} {S}_{ij} {v}_{j} = {b}_{i} & ∀ i ∈ I \\ {l}_{i} \leq {\sum }_{j ∈ J} {S}_{ij} {v}_{j} \leq {u}_{i} & ∀ i ∈ E \\ {α}_{j} \leq {v}_{j} \leq {β}_{j} & ∀ j ∈ J\end{matrix} \qquad [ Equation 4 ]$$

wherein Sᵢⱼ is the stoichiometric coefficient of the i^{th} metabolite in the j^{th} reaction, vⱼ is a metabolic flux vector in the j^{th} pathway, I is a set of all metabolites, J is a set of metabolic fluxes in all pathways, E is a set of all influent/effluent metabolic fluxes, cⱼ is a weighted value for a metabolic flux for the j^{th} pathway, bᵢ is the net transport flux of the i^{th} metabolite, lᵢ and uᵢ denote the net transport flux of the i^{th} metabolite, and αⱼ and βⱼ are limit values, which can be processed by metabolix flux in the j^{th} pathway.

In yet another aspect, the present invention provides a method for screening a gene to be amplified or deleted for increasing the production of a useful substance, the method comprising using the optimal value and profile of the overall metabolic flux of a metabolic network, plotted according to the above-described analysis method.

In yet another aspect, the present invention provides a method for improving E. coli producing a useful substance, the method comprising amplifying or deleting said screened gene in E. coli.

Other features and embodiments of the present invention will be more fully apparent from the following detailed description and appended claims.

### BRIEF DESCRIPTION OF DRAWINGS

FIG. 1 shows a metabolic flux analysis method according to the present invention.

FIG. 2 shows a method of defining CRD and SRD from a growth experiment using a radioactive isotope-labeled carbon source according to the present invention.

FIG. 3 shows a method of defining the correlation between relevant reaction equations using artificial metabolites on the basis of CRD and SRD defined according to the present invention.

FIG. 4 shows an example in which CRD and SRD are applied using MetaFluxNet according to the present invention.

FIG. 5 shows an example in which CRD and SRD are applied to the metabolic network model of E. coli using artificial metabolites, and shows the result values of the example (FIG. 5A) and the accuracy of the result values (FIG. 5B).

### DETAILED DESCRIPTION OF THE INVENTION, AND PREFERRED EMBODIMENTS THEREOF

As used herein, the term “correlation ratios” between specific metabolic fluxes refers to correlations, such as metabolic flux values resulting from various environmental changes or growth curves, or constant increases or decreases from all experimental numerical values predictable from the metabolic flux values, and is meant to include all conditions that can indicate the correlations.

In order to perform the improved metabolic flux analysis technique according to the present invention, a target organism (except for human beings), the overall metabolic flux of which is to be measured, is first selected, and the metabolic network model of the selected organism is constructed. Then, specific metabolic fluxes having correlation therebetween are identified based on the above-described experiments, whether a metabolic flux flowing into or flowing from a specific metabolite exists is determined to define the degree of contribution of the metabolic fluxes as CRD or SRD depending on the magnitude of the metabolic fluxes. Also, to measure the metabolic flux of the selected organism, experiments, including a growth experiment using a radioactive isotope-labeled carbon source and an assay for enzyme reaction are performed, and CRD and SRD is determined based on the experiment results. Then, the determined CRD and SRD are applied to a stoichiometric matrix using artificial metabolites, and the optimal value and profile of the overall metabolic flux are plotted on the basis of linear programming. The optimal value and profile of the overall metabolic flux can be calculated using the following algorithms:

$$Maximize \text{/} minimize \text{:} Z = {\sum }_{j ∈ J} {c}_{j} {v}_{j}$$

$$Subject to$$

$$\begin{matrix}{\sum }_{j ∈ J} {S}_{ij} {v}_{j} = {b}_{i} & ∀ i ∈ I \\ {l}_{i} \leq {\sum }_{j ∈ J} {S}_{ij} {v}_{j} \leq {u}_{i} & ∀ i ∈ E \\ {α}_{j} \leq {v}_{j} \leq {β}_{j} & ∀ j ∈ J\end{matrix}$$

wherein Sᵢⱼ is the stoichiometric coefficient of the i^{th} metabolite in the j^{th} reaction, vⱼ is a metabolic flux vector in the j^{th} pathway, I is a set of all metabolites, J is a set of metabolic fluxes in all pathways, E is a set of all influent/effluent metabolic fluxes, cⱼ is a weighted value for a metabolic flux for the j^{th} pathway, bᵢ is the net transport flux of the i^{th} metabolite, lᵢ and uᵢ denote the net transport flux of the i^{th} metabolite, and αⱼ and βⱼ are limit values, which can be processed by metabolic flux in the j^{th} pathway.

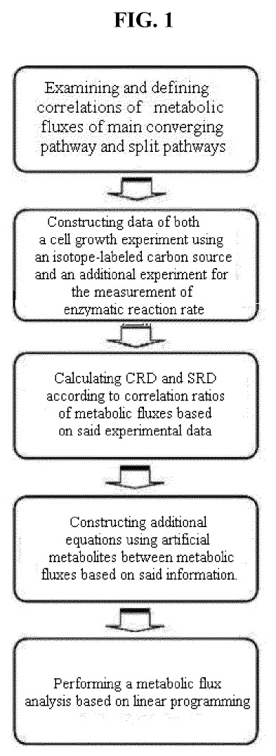

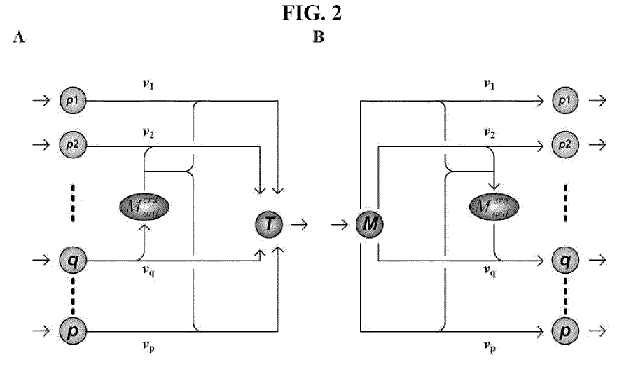

FIG. 1 shows an overall process for performing a method of plotting the overall metabolic flux profile with higher accuracy using the improved metabolic flux technique according to the present invention, and FIG. 2 schematically illustrate an example of applying said method to metabolic flux analysis.

To apply the improved metabolic analysis method according to the present invention, CRD and SRD for main converging pathways (A) or split pathways (B) should be first defined. From preliminary experiments, including an experiment of measuring the isotope labeling of typical metabolites using a radioactive isotope-labeled carbon source, and an assay for measuring enzyme reaction of main converging pathways or split pathways, the correlation ratios of specific metabolic fluxes, and f are determined, and CRD and SRD are defined based on the determined correlation ratios. Herein, the sum of the correlation ratios in the main split pathways or converging pathways is defined as 1:

$${\sum }_{p ∈ P} {f}_{p} = 1 , ∀ p ∈ P$$

wherein fₚ is the correlation ratio of specific metabolic flux p, P represents all the converging pathways or split pathways.

The following shows a mathematical representation for defining CRD for specific metabolic fluxes in converging pathways (FIG. 2A) from the exemplary model of FIG. 2 in order to apply the present invention:

$${C}_{q} = \frac{{\sum }_{p ∈ P} {f}_{p} - {f}_{q}}{{f}_{q}} , ∀ q ∈ P$$

wherein fₚ is the correlation ratio of specific metabolic flux p, f_{q} is the correlation ratio of specific metabolic flux q, C_{q} is CRD for specific metabolic flux v_{q} in converging pathways.

The following shows a mathematical representation for defining SRD for specific metabolic fluxes in split pathways (FIG. 2B) from the exemplary model of FIG. 2 in order to apply the present invention:

$${D}_{q} = \frac{{\sum }_{p ∈ P} {f}_{p} - {f}_{q}}{{f}_{q}} , ∀ p ∈ P$$

wherein fₚ is the correlation ratio of specific metabolic flux p, f_{q} is the correlation ratio of specific metabolic flux q, D_{q} is SRD for specific metabolic flux v_{q} in split pathways.

To apply the defined CRD or SRD to a stoichiometric matrix for solving linear programming, artificial metabolite M_{artf} between associated metabolic fluxes is defined as shown in FIG. 2. The CRD and SRD obtained through the above calculation procedure are applicable directly as stoichiometric coefficients for the artificial metabolite M_{artf}, the reaction equations thereof are as follows, and the sum thereof is 0, assuming a quasi-steady state:

$$\begin{matrix}\frac{d {M}_{artf}^{C R D}}{d t} = {input}_{{M}_{artf}} - {output}_{{M}_{artf}} \\ = {v}_{q} - {C}_{q} ( {\sum }_{p ∈ P} {v}_{p} - {v}_{q} ) \\ = 0\end{matrix}$$

$$\begin{matrix}\frac{d {M}_{artf}^{S R D}}{d t} = {input}_{{M}_{artf}} - {output}_{{M}_{artf}} \\ = {v}_{q} - {D}_{q} ( {\sum }_{p ∈ P} {v}_{p} - {v}_{q} ) \\ = 0\end{matrix}$$

wherein M_{artf}^{CRD} is an artificial metabolite for CRD in converging pathways, M_{artf}^{SRD} is an artificial metabolite for SRD in split pathways, v_{q} is the metabolic flux rate of metabolic flux q, C_{q} is CRD for specific metabolic flux v_{q}, and D_{q} is SRD for specific metabolic flux v_{q}. As a result, the reaction equations for the artificial metabolite are applied as equality constraints in linear programming.

CRD and SRD values can sometimes become values having a range between the maximum value and the minimum value according to various experimental errors. Thus, in this case, the reaction equations for the artificial metabolite will also have ranges, and are applied as inequality constraints, and the mathematical representations thereof are as follows:

$$\frac{d {M}_{artf}^{{C}^{min}}}{d t} = {v}_{q} - {C}_{q}^{min} ( {\sum }_{p ∈ P} {v}_{p} - {v}_{q} ) \geq 0$$

$$\frac{d {M}_{artf}^{{C}^{max}}}{d t} = {v}_{q} - {C}_{q}^{max} ( {\sum }_{p ∈ P} {v}_{p} - {v}_{q} ) \leq 0$$

wherein, M_{artf}^{C}^{^{min}} is minimum stoichiometric value of artificial metabolite M, M_{artf}^{C}^{^{max}} is maximum stoichiometric value of artificial metabolite M, Cqmin is minimum CRD value for v_{q}, Cqmax is maximum CRD value for v_{q}.

In the present invention, artificial metabolites are used to apply the above-defined CRD and SRD to a stoichiometric matrix for solving linear equations so as to effectively apply additional limit values, and the resulting stoichiometric matrix is used to perform the analysis of the overall metabolic flux on the basis of linear programming.

The following software systems can be used to calculate metabolic fluxes: Matlab-based software systems, such as FluxAnalyzer (Klamt et al., Bioinformatics, 19:216, 2003) and Metabologica (Zhu et al., Metab. Eng. 5:74, 2003), programs that can independently perform calculation, including Fluxor (http://arep.med.harvard.edu/moma/biospicefluxor.html), Simpheny (Genomatica Inc., San Diego, Calif.), INSILICO Discovery (INSILCO biotechnology Inc., Stuttgart, Germany), FBA (http://systemsbiology.used.edu/downloads/fba.htm), and MetaFluxNet (Lee et al., Bioinformatics, 19:2144, 2003), and computer language package tools, such as Gams (GAMS Development Corporation, NW Washington, D.C.), C language, and Fortran.

In the present invention, an E. coli model system was selected as a model system for applying said method, the analysis of overall metabolic flux of E. coli was performed from preliminary experiments.

### EXAMPLES

Hereinafter, the present invention will be described in more detail by specific examples. However, the present invention is not limited to these examples, and it is obvious to those skilled in the art that numerous variations or modifications could be made within the spirit and scope of the present invention.

In particular, the following examples illustrate performing an improved metabolic flux analysis using Escherichia coli as a strain, the metabolic flux of which is to be analyzed, it will be obvious to those skilled in the art from the disclosure of the present invention that strains other than E. coli are used for the analysis of metabolic flux.

Also, in the following examples, the correlation ratios of metabolic fluxes were determined by GC-MS (gas chromatography mass spectrometry) through an experiment using a radioactive isotope-labeled carbon source, as a preliminary experiment for defining CRD and SRD. However, it will be obvious to those skilled in the art from the disclosure of the present invention that all other experiments, including the measurement of enzymatic reaction rate, which can indicate the correlation between metabolic fluxes, can be used as experiments for defining CRD and SRD.

### Example 1

### Application of CRD and SRD in Exemplary Model

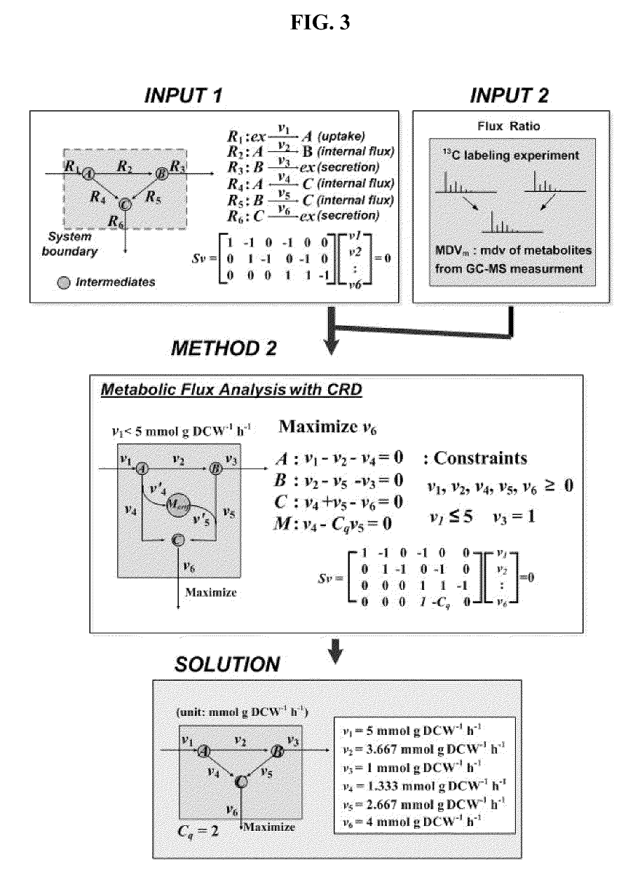

As an exemplary model for metabolic flux analysis, a system shown in FIG. 3 was constructed. The exemplary model included five reaction equations, three metabolites and one uptake (R1), and the maximum value of v6 as an objection function in metabolic flux analysis was set. Simulation was performed using MetaFluxNet 1.6, which could be downloaded through http://mbel.kaist.ac.kr/ (Lee et al., Bioinformatics, 19:2144, 2003).

For comparison, metabolic flux analysis, which does not include CRD and SRD, was primarily performed. When assuming a pseudo-steady state, the stoichiometric matrix of the exemplary model was as follows:

$$S \cdot v = [ \begin{matrix}1 & - 1 & 0 & - 1 & 0 & 0 \\ 0 & 1 & - 1 & 0 & - 1 & 0 \\ 0 & 0 & 0 & 1 & 1 & - 1\end{matrix} ] \cdot [ \begin{matrix}v 1 \\ v 2 \\ v 3 \\ v 4 \\ v 5 \\ v 6\end{matrix} ] = 0$$

Metabolic flux analysis was performed on the basis of the stoichiometric matrix according to linear programming. For a similar experiment of the input of experimental limit values, the metabolic rate of R3, one of external metabolic fluxes, was set at 1 mmol/g DCWh and inputted as limit value, the metabolic flux value of R1 as the uptake metabolic flux of the carbon source was set at 5 mmol/g DCWh, and the results were as follows:

Maximize v₆

A:v₁−v₃−v₄=0

v₁=5 mmol/g DCW h

B:v₂−v₅−v₃=0

v₂=5 mmol/g DCW h

C:v₄+v₅−v₆=0

v₃≦1 mmol/g DCW h

: constraints

v₄=0

v₁, v₂, v₄, v₅, v₆≧0

v₅=4 mmol/g DCW h

v₁≦5 v₃=1

v₆=4 mmol/g DCW h

This exemplary model included one converging pathway with respect to metabolite C, and reaction equations R4 and R5 for the converging pathway were set to have correlation. Also, CRD, C_{q}, according to the correlation ratio, was set at 0.5.

To apply CRD, artificial metabolite M_{artf} between the two metabolic fluxes having correlation was set, and FIG. 3 shows a method of applying CRD to this model. Also, according to the present invention, the coefficient of the artificial metabolite was defined as Dq=0.5, which was CRD value. The resulting modified stoichiometric matrix was as follows:

$$S \cdot v = [ \begin{matrix}1 & - 1 & 0 & - 1 & 0 & 0 \\ 0 & 1 & - 1 & 0 & - 1 & 0 \\ 0 & 0 & 0 & 1 & 1 & - 1 \\ 0 & 0 & 0 & 1 & - {C}_{q} & 0\end{matrix} ] \cdot [ \begin{matrix}v 1 \\ v 2 \\ v 3 \\ v 4 \\ v 5 \\ v 6\end{matrix} ] = 0$$

Metabolic flux analysis was performed on the basis of the stoichiometric matrix according to linear programming. For a similar experiment of the input of experimental limit values, the metabolic rate of R3, one of external metabolic fluxes, was set at 1 mmol/g DCWh and inputted as limit value, and the metabolic flux value of R1 as the uptake metabolic flux of the carbon source was set at 5 mmol/g DCWh. The results were as follows:

Maximize v₆

A:v₁−v₂−v₄=0

B:v₂−v₅−v₃=0

C:v₄+v₅−v₆=0

M:v₄+C_{q}v₅=0

: constraints

v₁, v₂, v₄, v₅, v₆≧0 v₁≦5 v₃=1

v₁=5 mmol/g DCW h

v₂=3.667 mmol/g DCW h

v₃=1 mmol/g DCW h

v₄=1.333 mmol/g DCW h

v₅=2.667 mmol/g DCW h

v₆=4 mmol/g DCW h

As shown in FIG. 3, the example of general metabolic flux analysis, to which CRD was not applied, showed a simple combination of internal metabolic flux distributions, and particularly, R4, one of the converging pathways for the metabolite C, did not have the value thereof. Obviously, this is a phenomenon, which frequently appears in metabolic flux distribution for several converging pathways or split pathways in performing metabolic flux analysis using the metabolic network of actual cells according to linear programming, and is clearly not an actual phenomenon.

However, unlike the above case, in metabolic flux distribution obtained by defining the correlation ratio between two metabolic fluxes constituting converging pathways as CRD and the artificial metabolite, the metabolic flux distribution of the converging pathways was distinguished according to the correlation ratio, even though the value of the objective function was the same. Because this correlation ratio was on the basis of experiments, it showed an excellent effect of showing metabolic flux distribution more close to an actual phenomenon.

### Example 2

### Example of Application of CRD in Metabolic Network Model of E. coli

In the case of E. coli, a new metabolic network consisted of 979 biochemical reactions, and 814 metabolites were considered on the metabolic network. This system included almost all of the biochemical reactions of E. coli, and the biomass composition of E. coli for forming a biomass formation equation to be used as an objective function was as follows (Neidhardt et al., Cellular and Molecular Biology, 1996): 55% protein, 20.5% RNA, 3.1% DNA, 9.1% lipids, 3.4% lipopolysaccharides, 2.5% peptidoglycan, 2.5% glycogen, 0.4% polyamines, and 3.5% other metabolites, cofactors and ions.

Generally, E. coli seems to grow using cell constituents at the maximum, and this is expressed as specific growth rate. Thus, metabolic flux analysis was performed according to linear programming using the specific growth rate as an objective function.

First, in E. coli, experimental values that can define correlation ratio value were obtained from an experiment using a radioactive isotope-labeled carbon source. To determine the correlation ratio between metabolic fluxes from GCMS (gas chromatography-mass spectrometry) measurement, the method of E. Fischer et al. (E. Fischer et al., Eur. J. Biochem. 270:880, 2003) was performed, and the mathematical representations thereof are as follows.

MDV (mass distribution vector) was defined according to the isotope contents of main metabolites, modified by removing the measurement values of the amounts of natural radioactive isotopes, and the sum of the elements of each metabolite was defined as 1:

$$MDV = [ \begin{matrix}( {m}_{0} ) \\ ( {m}_{1} ) \\ ( {m}_{2} ) \\ … \\ ( {m}_{n} )\end{matrix} ] with \sum {m}_{i} = 1$$

The isotope contents of the elements of metabolites located in main converging pathways are based on the isotope contents of precursors thereto, and depend on the magnitudes of metabolic fluxes based on the metabolic flux network, and particularly, the sum of the elements of MDV that defines the isotope content of each metabolite is 1. Thus, from the MDV of each metabolite, the correlation ratio between metabolic fluxes, which are located in converging pathways and associated with each other, is defined as follows:

$${f}_{p 1} = \frac{{MDV}_{M} - {MDV}_{p 2}}{{MDV}_{p 1} - {MDV}_{p 2}}$$

wherein M is a target metabolite present in converging pathways, p1 and p2 are metabolites serving as precursors of the metabolite M, and fₚ₁ denotes the correlation ratio of a metabolic flux from p1 to a metabolic flux from p2. Herein, the sum of the correlation ratios of the associated metabolic fluxes is defined as 1, and the mathematical representation thereof is as follows:

$${\sum }_{p ∈ P} {f}_{p} = 1 , ∀ p ∈ P$$

To apply the present invention, CRD for specific metabolic fluxes was defined and the mathematical representation thereof was as follows:

$${C}_{q} = \frac{{\sum }_{p ∈ P} {f}_{p} - {f}_{q}}{{f}_{q}} , ∀ q ∈ P$$

wherein fₚ is the correlation ratio of specific metabolic flux p, f_{q} is the correlation ratio of specific metabolic flux q, C_{q} is CRD for specific metabolic flux q in converging pathways.

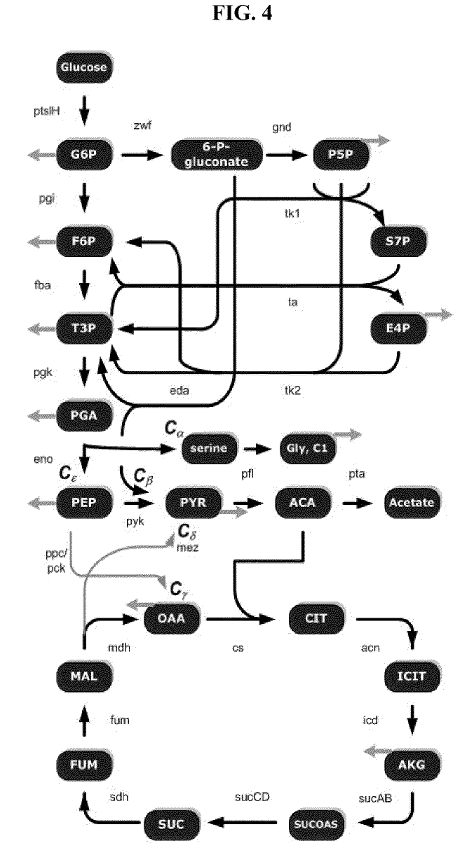

In the metabolic flux network of E. coli, the correlations between five converging pathways in main central metabolic flux network, including glycolysis, phentosphosphate pathway, TCA and anaplerotic pathway, were defined, and expressed in the names of the target metabolites and reaction equations of the main converging pathways as shown in FIG. 4. To apply the correlation ratios of the associated metabolic fluxes from experimental data, additional reaction equations were defined using artificial metabolites between the associated metabolic fluxes, and when assuming a quasi-steady state, a reaction equation for each artificial metabolite was as follows.

$$\frac{d {M}_{α}}{d t} = 2 ( {v}_{fba} - {v}_{tk 2} - {v}_{ta} ) - {C}_{α} ( {v}_{eda} + {v}_{tk 1} + 3 {v}_{tk 2} + 2 {v}_{ta} ) = 0$$

$$\frac{d {M}_{ρ}}{d t} = {v}_{eda} - {C}_{ρ} ( {v}_{pyk} + {v}_{mez} ) = 0$$

$$\frac{d {M}_{γ}}{d t} = {v}_{ppc} - {C}_{γ} {v}_{mdh} = 0$$

$$\frac{d {M}_{δ}^{{C}^{max}}}{d t} = {v}_{mez} - {C}_{δ}^{max} ( {v}_{eda} + {v}_{pyk} ) \leq 0$$

$$\frac{d {M}_{δ}^{{C}^{min}}}{d t} = {v}_{mez} - {C}_{δ}^{min} ( {v}_{eda} + {v}_{pyk} ) \geq 0$$

$$\frac{d {M}_{τ}^{{C}^{max}}}{d t} = ( {v}_{tk 1} + 3 {v}_{tk 2} + 2 {v}_{ta} ) - {C}_{τ}^{max} ( \begin{matrix}{v}_{eda} + 2 {v}_{fba} - \\ 2 {v}_{tk 2} - 2 {v}_{ta}\end{matrix} ) \leq 0$$

wherein, referring to FIG. 4, V_{fba} is the amount of flux of a fba reaction from F6P to T3P, vₜₖ₂ is the amount of flux of a tk2 reaction from E4P and P5P to F6P and T3P, vₜₖ₁ is the amount of flux of a tk1 reaction from P5P to S7P and T3P, vₜₐ is the amount of flux of a ta reaction from S7P and T3P to F6P and E4P, v_{ppc} is the amount of flux of a ppc reaction from PEP to OAA, v_{eda} is the amount of flux of an eda reaction from 6-P-gluconate to T3P and PYR, v_{mdh} is the amount of flux of an mdh reaction from MAL to OAA, v_{pyk} is the amount of flux of a pyk reaction from PEP to PYR, v_{mez} is the amount of flux of an mez reaction from MAL to PYR, C_{α} and M_{α} represent CRD and an artificial metabolite, respectively, for specific converging pathway α, C_{β} and M_{β} represent CRD and an artificial metabolite, respectively, for specific converging pathway β, C_{γ} and M_{γ} represent CRD and an artificial metabolite, respectively, for specific converging pathway γ, and C_{δ} and M_{δ} represent CRD and an artificial metabolite, respectively, for specific converging pathway δ.

A total of five artificial metabolites for five converging pathways were determined, the reaction equations therefor were defined, and three equality constraints and two inequality constraints were defined depending on whether the correlation ratio values according to the experimental values had ranges.

The MDV value of each metabolite, resulting from actual experiments for determining each CRD value in a reaction equation for each artificial metabolite, and correlation ratio resulting from the MDV value, were calculated according to the experimental data of Fischer et al. (Anal. Biochem. 325:308, 2004) and conformed to experimental numerical values obtained using a 20/80% [U-13C]glucose/[1-13C]glucose-labeled carbon source. CRD values defined according to the above method are shown in Table 1 below.

TABLE 1

| Artificial metabolites | Converging ratio determinant (C) |
|---|---|
| Mₐ for serine | 2.57 ± 0.06 |
| M_{b} for pyruvate | 0.01 ± 0.10 |
| M_{g} for oxaloacetate | 1.86 ± 0.06 |
| M_{d} for pyruvate (upper bound) | 0.19 ± 0.14 |
| M_{d} for pyruvate (lower bound) | 0.06 ± 0.04 |
| Mₑ for phosphoenolpyruvate | 0.20 ± 0.10 |
| (upper bound) | |

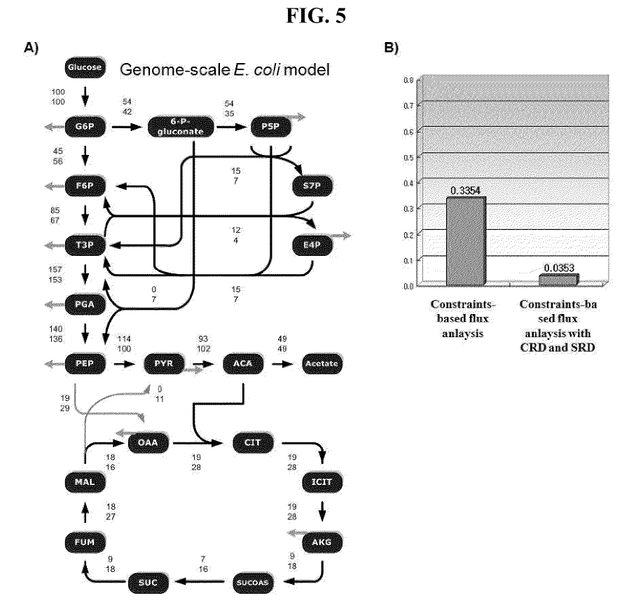

As described above, while the additionally defined reaction equations and CRD values for artificial metabolites were applied, the metabolic flux analysis of E. coli was performed according to linear programming assuming a quasi-steady state. Among the analysis results, an example of metabolic flux distribution in the main central metabolic network is shown in FIG. 5. Also, based on metabolic flux distributions obtained for the main central metabolic flux network according to non-linear programming, the accuracy of the result of use of the present invention (upper value in FIG. 5A) and the result of non-use of the present invention (lower value in FIG. 5B) is shown in FIG. 5B.

On the basis of the above results, it could be found that, when several additional numerical values obtained through additional experiments were effectively applied as additional limit values to linear programming, more accurate intracellular metabolic flux distribution could be obtained.

### Example 3

### Gene Screening and Organism Improvement for Increasing Production of Useful Substance Using the Present Invention

To increase the production of a useful substance, a gene to be amplified, which increase the production of the useful substance, was screened using the optimal value and profile of the overall metabolic flux, obtained according to Examples 1 and 2. The screening of the gene to be amplified was performed according to the method described in Korean Patent Application No. 10-2005-0086119.

Also, the gene to be amplified, screened according to the method described in Korean Patent Application No. 10-2005-0086119, can be introduced or amplified in the relevant organism to construct a mutant of the relevant organism.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof. Those skilled in the art will appreciate that simple modifications, variations and additions to the present invention are possible, without departing from the scope and spirit of the invention as disclosed in the accompanying claims.

### INDUSTRIAL APPLICABILITY

As described in detail above, the present invention provides the metabolic flux analysis method and the method for analyzing the metabolic flux of E. coli. According to the present invention, the correlation between influent/effluent metabolic fluxes with respect to specific metabolites in a target organism, a genome-scale metabolic network model of which was constructed, can be determined as relative ratio using useful information obtained from various experiments, including a growth experiment using a radioactive isotope-labeled carbon source and an experiment for measuring enzymatic reaction rate. Thus, limit values from various experiments can be effectively applied, so that internal metabolic flux can be quantified and analyzed in a more accurate and rapid manner.

1. A method for analyzing the metabolic flux of a target organism, the method comprising the steps of:
(a) selecting a target organism (except for human beings), the overall metabolic flux of which is to be measured, and constructing the metabolic network model of the selected organism;
(b) identifying the correlations between specific metabolic fluxes in the metabolic network model of the step (a), and defining the correlation ratios of the specific metabolic fluxes as CRD (converging ratio determinant) and SRD (split ratio determinant);
(c) performing the experiment for measuring metabolic flux ratios on the selected organism, and determining and correcting the correlation ratios, CRD and SRD of the step (b) on the basis of the experimental results;
(d) defining artificial metabolites between the specific metabolic fluxes;
(e) determining additional equations by applying the corrected correlation ratios, CRD and SRD of the step (c) to a stoichiometric matrix for solving linear equations using the artificial metabolites of the step (d), and applying the determined additional equations to the stoichiometric matrix to modify the stoichiometric matrix; and
(f) plotting the optimal value and profile of the overall metabolic flux of the metabolic network according to linear programming using the stoichiometric matrix modified in the step (e).

2. The method for analyzing the metabolic flux of a target organism according to claim 1, wherein the target organism is a microorganism.

3. The method for analyzing the metabolic flux of a target organism according to claim 1, wherein the correlation ratios of the specific metabolic fluxes in the step (b) are defined as CRD and SRD using the following equations 1 and 2:
$$\begin{matrix}{C}_{q} = \frac{{\sum }_{p ∈ P} {f}_{p} - {f}_{q}}{{f}_{q}} , ∀ q ∈ P & [ Equation 1 ] \\ {D}_{q} = \frac{{\sum }_{p ∈ P} {f}_{p} - {f}_{q}}{{f}_{q}} , ∀ q ∈ P & [ Equation 2 ]\end{matrix}$$
wherein fₚ is the correlation ratio of specific metabolic flux p, f_{q} is the correlation ratio of specific metabolic flux q, C_{q} is CRD for specific metabolic flux v_{q} in converging pathways, D_{q} is SRD for specific metabolic flux v_{q} in split pathways (provided that
$${\sum }_{p ∈ P} {f}_{p} = 1 , ∀ p ∈ P$$
wherein P represents all the converging pathways or split pathways).

4. The method for analyzing the metabolic flux of a target organism according to claim 1, wherein the experiment for measuring metabolic flux ratios in the step (c) is a growth experiment using a radioactive isotope-labeled carbon source and/or an experiment for the assay of enzymatic reaction.

5. The method for analyzing the metabolic flux of a target organism according to claim 1, wherein the artificial metabolites in the step (d) are defined using the following equation 3:
$$\begin{matrix}\frac{d {M}_{artf}^{C R D}}{d t} = {input}_{{M}_{artf}} - {output}_{{M}_{artf}} \\ = {v}_{q} - {C}_{q} ( {\sum }_{p ∈ P} {v}_{p} - {v}_{q} ) \\ = 0\end{matrix} \\ \begin{matrix}\frac{d {M}_{artf}^{S R D}}{d t} = {input}_{{M}_{artf}} - {output}_{{M}_{artf}} \\ = {v}_{q} - {D}_{q} ( {\sum }_{p ∈ P} {v}_{p} - {v}_{q} ) \\ = 0\end{matrix} \qquad [ Equation 3 ]$$
wherein M_{artf}^{CRD} is an artificial metabolite for CRD in converging pathways, M_{artf}^{SRD} is an artificial metabolite for SRD in split pathways, input_{Marf} is the sum of metabolic fluxes flowing into artificial metabolite M, output_{Marf} is the sum of metabolic fluxes flowing from artificial metabolite M, vₚ and v_{q} are the metabolic flux rates of metabolic fluxes p and q, respectively, C_{q} is CRD for specific metabolic flux q, and D_{q} is SRD for specific metabolic flux v_{q}.

6. The method for analyzing the metabolic flux of a target organism according to claim 1, wherein the stoichiometric matrix before modification in the step (e) is represented as follows:
$$S \cdot v = [ \begin{matrix}1 & - 1 & 0 & - 1 & 0 & 0 \\ 0 & 1 & - 1 & 0 & - 1 & 0 \\ 0 & 0 & 0 & 1 & 1 & - 1\end{matrix} ] \cdot [ \begin{matrix}v 1 \\ v 2 \\ v 3 \\ v 4 \\ v 5 \\ v 6\end{matrix} ] = 0$$

7. The method for analyzing the metabolic flux of a target organism according to claim 1, wherein the stoichiometric matrix after modification in the step (e) is represented as follows:
$$S \cdot v = [ \begin{matrix}1 & - 1 & 0 & - 1 & 0 & 0 \\ 0 & 1 & - 1 & 0 & - 1 & 0 \\ 0 & 0 & 0 & 1 & 1 & - 1 \\ 0 & 0 & 0 & 1 & - {C}_{q} & 0\end{matrix} ] \cdot [ \begin{matrix}v 1 \\ v 2 \\ v 3 \\ v 4 \\ v 5 \\ v 6\end{matrix} ] = 0$$

8. The method for analyzing the metabolic flux of a target organism according to claim 1, wherein the optimal value and profile of the overall metabolic flux in the step (f) are plotted using the following equation 4:
$$Maximize \text{/} minimize \text{:} Z = {\sum }_{j ∈ J} {c}_{j} {v}_{j} \\ Subject to \\ \begin{matrix}{\sum }_{j ∈ J} {S}_{ij} {v}_{j} = {b}_{i} & ∀ i ∈ I \\ {l}_{i} \leq {\sum }_{j ∈ J} {S}_{ij} {v}_{j} \leq {u}_{i} & ∀ i ∈ E \\ {α}_{j} \leq {v}_{j} \leq {β}_{j} & ∀ j ∈ J\end{matrix} \qquad [ Equation 4 ]$$
wherein Sᵢⱼ is the stoichiometric coefficient of the i^{th} metabolite in the j^{th} reaction, vⱼ is a metabolic flux vector in the j^{th} pathway, I is a set of all metabolites, J is a set of metabolic fluxes in all pathways, E is a set of all influent/effluent metabolic fluxes, cⱼ is a weighted value for a metabolic flux for the j^{th} pathway, bᵢ is the net transport flux of the i^{th} metabolite, lᵢ and uᵢ denote the net transport flux of the i^{th} metabolite, and αⱼ and βⱼ are limit values, which can be processed by a metabolic flux in the j^{th} pathway.

9. A method for screening a gene to be amplified or deleted for increasing the production of a useful substance, the method comprising using the optimal value and profile of the overall metabolic flux of a metabolic network, plotted by the method of claim 1.

10. A method for improving an organism producing a useful substance, the method comprising amplifying or deleting the gene screened by the method of claim 9, in a target organism.

11. A method for analyzing the metabolic flux of E. coli, comprising the steps of:
(a) constructing a metabolic network model of E. coli;
(b) identifying the correlations between specific metabolic fluxes in the metabolic network model constructed in the step (a), and defining the correlation ratios of the specific metabolic fluxes as CRD (converging ratio determinant) and SRD (split ratio determinant);
(c) performing the experiment for measuring metabolic flux ratios on the E. coli, and determining and correcting the correlation ratios, CRD and SRD of the step (b) on the basis of the experimental results;
(d) defining artificial metabolites between the specific metabolic fluxes;
(e) determining additional equations by applying the corrected correlation ratios, CRD and SRD of the step (c) to a stoichiometric matrix for solving linear equations using the artificial metabolites of the step (d), and applying the determined additional equations to the stoichiometric matrix to modify the stoichiometric matrix; and
(f) plotting the optimal value and profile of the overall metabolic flux of the metabolic network according to linear programming using the stoichiometric matrix modified in the step (e).

12. The method for analyzing the metabolic flux of E. coli according to claim 11, wherein the correlation ratios of the specific metabolic fluxes in the step (b) are defined as CRD and SRD using the following equations 1 and 2:
$$\begin{matrix}{C}_{q} = \frac{{\sum }_{p ∈ P} {f}_{p} - {f}_{q}}{{f}_{q}} , ∀ q ∈ P & [ Equation 1 ] \\ {D}_{q} = \frac{{\sum }_{p ∈ P} {f}_{p} - {f}_{q}}{{f}_{q}} , ∀ q ∈ P & [ Equation 2 ]\end{matrix}$$
wherein fₚ is the correlation ratio of specific metabolic flux p, f_{q} is the correlation ratio of specific metabolic flux q, C_{q} is CRD for specific metabolic flux v_{q} in converging pathways, D_{q} is SRD for specific metabolic flux v_{q} in split pathways (provided that
$${\sum }_{p ∈ P} {f}_{p} = 1 , ∀ p ∈ P$$
wherein P represents all the converging pathways or split pathways).

13. The method for analyzing the metabolic flux of E. coli according to claim 11, wherein the metabolic flux measurement experiment in the step (c) is a growth experiment and/or an experiment for enzymatic reaction rate measurement using a radioactive isotope-labeled carbon source.

14. The method for analyzing the metabolic flux of E. coli according to claim 11, wherein the artificial metabolites in the step (d) are defined using the following equation 3:
$$\begin{matrix}\frac{d {M}_{artf}^{C R D}}{d t} = {input}_{{M}_{artf}} - {output}_{{M}_{artf}} \\ = {v}_{q} - {C}_{q} ( {\sum }_{p ∈ P} {v}_{p} - {v}_{q} ) \\ = 0\end{matrix} \\ \begin{matrix}\frac{d {M}_{artf}^{S R D}}{d t} = {input}_{{M}_{artf}} - {output}_{{M}_{artf}} \\ = {v}_{q} - {D}_{q} ( {\sum }_{p ∈ P} {v}_{p} - {v}_{q} ) \\ = 0\end{matrix} \qquad [ Equation 3 ]$$
wherein M_{artf}^{CRD} is an artificial metabolite for CRD in converging pathways, M_{artf}^{SRD} is an artificial metabolite for SRD in split pathways, input_{Marf} is the sum of metabolic fluxes flowing into artificial metabolite M, output_{Marf} is the sum of metabolic fluxes flowing from artificial metabolite M, vₚ and v_{q} are the metabolic flux rates of metabolic fluxes p and q, respectively, C_{q} is CRD for specific metabolic flux q, and D_{q} is SRD for specific metabolic flux v_{q}.

15. The method for analyzing the metabolic flux of E. coli according to claim 11, wherein the stoichiometric matrix before modification in the step (e) is represented as follows:
$$S \cdot v = [ \begin{matrix}1 & - 1 & 0 & - 1 & 0 & 0 \\ 0 & 1 & - 1 & 0 & - 1 & 0 \\ 0 & 0 & 0 & 1 & 1 & - 1\end{matrix} ] \cdot [ \begin{matrix}v 1 \\ v 2 \\ v 3 \\ v 4 \\ v 5 \\ v 6\end{matrix} ] = 0$$

16. The method for analyzing the metabolic flux of E. coli according to claim 11, wherein the stoichiometric matrix after modification in the step (e) is represented as follows:
$$S \cdot v = [ \begin{matrix}1 & - 1 & 0 & - 1 & 0 & 0 \\ 0 & 1 & - 1 & 0 & - 1 & 0 \\ 0 & 0 & 0 & 1 & 1 & - 1 \\ 0 & 0 & 0 & 1 & - {C}_{q} & 0\end{matrix} ] \cdot [ \begin{matrix}v 1 \\ v 2 \\ v 3 \\ v 4 \\ v 5 \\ v 6\end{matrix} ] = 0$$

17. The method for analyzing the metabolic flux of E. coli according to claim 11, wherein the optimal value and profile of the overall metabolic flux in the step (f) are plotted using the following equation 4:
$$Maximize \text{/} minimize \text{:} Z = {\sum }_{j ∈ J} {c}_{j} {v}_{j} \\ Subject to \\ \begin{matrix}{\sum }_{j ∈ J} {S}_{ij} {v}_{j} = {b}_{i} & ∀ i ∈ I \\ {l}_{i} \leq {\sum }_{j ∈ J} {S}_{ij} {v}_{j} \leq {u}_{i} & ∀ i ∈ E \\ {α}_{j} \leq {v}_{j} \leq {β}_{j} & ∀ j ∈ J\end{matrix} \qquad [ Equation 4 ]$$
wherein Sᵢⱼ is the stoichiometric coefficient of the i^{th} metabolite in the j^{th} reaction, vⱼ is a metabolic flux vector in the j^{th} pathway, I is a set of all metabolites, J is a set of metabolic fluxes in all pathways, E is a set of all influent/effluent metabolic fluxes, cⱼ is a weighted value for a metabolic flux for the j^{th} pathway, bᵢ is the net transport flux of the i^{th} metabolite, lᵢ and uᵢ denote the net transport flux of the i^{th} metabolite, and αⱼ and βⱼ are limit values, which can be processed by a metabolic flux in the j^{th} pathway.

18. A method for screening a gene to be amplified or deleted for increasing the production of a useful substance, the method comprising using the optimal value and profile of the overall metabolic flux of a metabolic network, plotted by the method of claim 11.

19. A method for improving E. coli producing a useful substance, the method comprising amplifying or deleting the gene screened by the method of claim 18, in E. coli.
